(19) United States
(12) Patent Application Publication
(10) Pub. No.: US 2023/0338703 A1
(43) Pub. Date: Oct. 26, 2023

(54) ACCESS VESSEL FOR PATIENTS CONNECTED TO A FLUID CIRCUIT

(71) Applicants: ASIF K. MUSTAFA, Willowbrook, IL (US); DEVANG J. JOSH, Carol Stream, IL (US)

(72) Inventors: ASIF K. MUSTAFA, Willowbrook, IL (US); DEVANG J. JOSH, Carol Stream, IL (US)

(21) Appl. No.: 17/714,961

(22) Filed: Apr. 6, 2022

(51) Int. Cl. A61M 25/00

(52) U.S. Cl. CPC A61M 25/007; A61M 2025/1052

(57) ABSTRACT

This invention relates to the infusion of medications and/or fluids into a patient, and/or the removal of blood samples therefrom. More specifically, this invention relates to an apparatus, system and method of medication/fluid infusion into a patient and/or blood removal from a patient on an extracorporeal membrane oxygenation (ECMO) and/or cardiopulmonary bypass (CPB) fluid circuit such that the need for utilizing a central line, as well as HD or CRRT lines on the patient is avoided.

### CROSS REFERENCE TO RELATED APPLICATIONS

None

### TECHNICAL FIELD OF THE INVENTION

This invention relates to the infusion of medications and/or fluids into a patient, and/or the removal of blood samples therefrom. More specifically, this invention relates to an apparatus, system and method of medication/fluid infusion into a patient and/or blood removal from a patient on an extracorporeal membrane oxygenation (ECMO) and/or cardiopulmonary bypass (CPB) fluid circuit such that the need for utilizing a central line on the patient is avoided, thus avoiding or minimizing the associated bloodstream infections and numerous other disadvantages associated with central line use. Additionally, this invention obviates any need for dialysis catheters as well. Finally, the invention permits for a safe and rapid access for endovascular procedures.

### BACKGROUND OF THE INVENTION

Patients requiring the use of ECMO due to cardiac and/or respiratory failure utilize this treatment for extended periods of time. Given that they are critically ill, the need for central venous access at all times for infusions of medication and/or fluids (for treatment), and/or removal of blood samples (for laboratory analysis), and/or administration of dialysis (due to renal failure) is necessary. Additionally, patients who undergo surgery and require CPB also benefit from central venous access for the quick delivery of medications and/or fluids and/or removal of blood samples for analysis. Moreover, central vascular access for various other procedures/monitoring performed on an urgent or continuous basis is of great importance. These may include, without limitation: imaging, including pulmonary and peripheral angiographies; hemodynamic monitoring/cardiac function analysis, including right and left heart catheterization and intermittent or continuous pressure monitoring; transvenous pacemaker insertions; and any other endovascular procedures, such as embolization etc.

However, central venous access and dialysis catheters each pose a significant risk to the patient. Central venous access and dialysis catheterization are each possible via the insertion of a central line into the patient. This central line, also known as a central venous catheter, is a catheter or tube placed by medical practitioners within the large veins of a patient's neck, chest or groin for providing medications and/or fluids to a patient during critical or intensive care periods, as well as removing and sampling blood from the patient for laboratory analysis. Central lines generally differ from peripheral intravenous catheters (i.e., PIVs), which typically provide lower volumes of medications and/or liquids into a vein located near the skin's surface (i.e., on the arm or the hand), because of their location proximal to the heart and their ability to remain in place for longer time durations.

However, the foregoing use of central lines is fraught with disadvantages. A primary disadvantage is the risk of a patient contracting a central line associated bloodstream infection (i.e., CLABSI); which occurs when bacteria, viruses and/or other germs enter the blood stream through the central line and/or insertion site. To avoid the occurrence of CLABSI, medical practitioners must follow strict protocols to ensure that both the central line and insertion site on the patient remains sterile when inserting the line. Medical practitioners must also follow stringent infection control practices when checking and/or replacing the line, as well as changing the associated dressing (i.e., bandages, etc.).

Although numerous practices exist that promote sterile central line placement and maintenance, namely, hand hygiene practices and the use of sterile barrier precautions, to include sterile gloves, gowns, caps, masks and drapes, these practices and precautions are both time consuming and inconsistent between different medical practitioners. Of course, during a given medical emergency or critical care scenario, time is of the essence; where mere minutes may affect the success of a patient's medical outcome when trying to insert a central line for the infusion of critical medications and/or fluids into a patient. The insertion of these lines usually requires the assimilation of instruments (i.e. ultrasound), drapes, preps, etc., with the central line kit having many components that make it cumbersome, time consuming and expensive. Furthermore, only specially-trained practitioners can place these lines, usually in an intensive care unit, or an emergency or operating room setting.

Another disadvantage present with the use of central lines is the potential for injury to a patient's surrounding anatomical structures, i.e., blood vessels, nerves, etc., during its placement within the body. For example, placement of the line via a central venous access in the internal jugular vein can lead to injury of the carotid artery; placement via a subclavian approach can lead to injury of the subclavian artery or result in pneumothoraces due to lung injury; and placement via a femoral approach can lead to injury of femoral arteries, nerves, and bowel (possibly resulting in life-threatening retroperitoneal hematomas or bowel perforations).

Air embolus, while uncommon, also may result in a catastrophic event during central line placement. All of these complications are further exacerbated in obese individuals, where line placement becomes severely challenging due to body habitus. Moreover, central lines may also present a risk of pseudoaneurysm or arterio-venous fistula formation, requiring extensive tissue repair. Their long-term use may also create stenotic regions within the vessels, thus dangerously limiting blood flow (venous stenosis) there-through. Similarly, upper body central lines may cause superior vena cava syndrome leading to impaired venous blood return to the heart.

Yet other disadvantages relate to the insertion process utilized during such placement. A guide-wire, utilized for guiding the line into a given blood vessel, can be lost within the vessel itself, thereafter requiring the execution of yet further time-consuming procedures to retrieve the wire from therein. Also, the existence of another line or ECMO cannula within the patient creates locational difficulties when placing these central lines, thus requiring the assistance of other modalities, such as fluoroscopy. Also, in addition to utilizing ECMO, if the patient is anti-coagulated with medications and/or has pre-existing bleeding diathesis, the likelihood of having bleeding complications from line insertion is significantly greater. Furthermore, an incorrect placement of the original central line (e.g. terminating in the contralateral internal jugular vein instead of the superior vena cava) would require that the line be repositioned or replaced, again adding to the risk of the original procedure. Additionally, central lines commonly have a lifespan of approximately 7-10 days, depending on institution protocols, after which replacement of the line becomes necessary due to risk of the aforementioned central line-associated bloodstream infection (CLABSI) occurring.

A further disadvantage of central lines relates to limitations presented by their physical structure. The lines generally utilize infusion ports that define small bore diameters (creating fluid flow or pressure restrictions there-though) and/or are limited in quantity (i.e., a maximum of four ports). This limits both the number of different infusions that can be performed due to medication compatibility restrictions, and the rate at which they can be administered. Furthermore, central lines have malfunctioned due to either a kinking of the line or a thrombus formation (blood clot), thus necessitating manipulation or replacement of the line itself. Deep venous clots may lead to pulmonary emboli which may be life-threatening. Finally, patient comfort is an important factor. Despite the use of a local anesthetic, central line placement often proves to be a painful or traumatic experience for the patient. Moreover, having a central line in the neck, chest, or groin of a patient limits the patient's mobility and comfort throughout his or her hospital stay and poses a source of infection thereafter.

Additionally, similar problems are encountered when running a hemodialysis (HI)) machine or continuous renal replacement therapy (CRRT) in cases of renal failure in critically ill hospitalized patients on ECMO. Current techniques to perform HD or CRRT on ECMO patients rely on the placement of peripherally-inserted central dialysis catheters. Not only do these catheters have the same disadvantages of central lines, such as infection, thrombus formation etc they are highly prone to obstruction/occlusion due to the dialysis catheter's small caliber; thus requiring anti-coagulants or line replacement.

Finally, obtaining vascular access for procedures such as endovascular imaging, hemodynamic monitoring/cardiac function analysis, transvenous pacing, or any other endovascular procedures are fraught with the same hazards as with obtaining central line access. And given that such procedures often require the placement of a large bore central access catheter or cannula, the dangers are even greater.

Thus, what is needed are an apparatus, system and method for infusing patients with the greater volumes of medications and/or fluids possible with central line use, as well as for removing blood samples from patients for analysis, without the need for inserting the central line itself. The apparatus, system and method should also allow for establishing HD/CRRT connections into a much larger flow channel, thus preventing any pediments to the ITD/CRRT flow due to their smaller diameter. The apparatus, system and method should further allow for exclusive external access because nothing is inserted into the patient and there is no remnant foreign object (i.e., catheter guide-wire) of concern to medical practitioners. Given that the continuous and high-volume flow of ECMO circuits minimizes bloodstream infections compared to central line placements, the apparatus, system and method should allow for long term vascular access without requiring any apparatus replacement. Moreover, the apparatus, system and method should also accommodate the simultaneous infusion of multiple medications and/or fluids, and/or the simultaneous removal of blood samples for laboratory analysis. Finally, the apparatus, system and method should eliminate or minimize blood loss through an infusion/extraction point via a lowering of its associated internal fluid (i.e., blood) pressure. The present invention satisfies these foregoing disadvantages and presents other advantages as well.

### SUMMARY OF THE INVENTION

This invention relates to the infusion of medications and/or fluids into a patient, and/or the removal of blood samples therefrom. More specifically, this invention relates to an apparatus, system and method of administering such medication/fluid infusion into a patient and/or blood removal from a patient on extracorporeal membrane oxygenation (ECMO) and/or cardiopulmonary bypass (CPB) fluid circuits such that the need for utilizing central lines on the patient is avoided, thus also avoiding or minimizing the associated bloodstream infections and numerous other disadvantages associated with central line use and other means of vascular access. Additionally, this invention obviates any need for dialysis catheters as well. Finally, the invention permits for a safe and rapid access for endovascular procedures.

A system for infusing medications and/or fluids, and/or removing blood samples from a patient comprises a pump configured for fluid communication with the patient, and an oxygenator in fluid communication with the pump and configured for fluid communication with the patient, with the pump, oxygenator and associated fluid communications comprising a fluid circuit. The fluid circuit is utilized in a medical setting to receive the blood of the patient, oxygenate it, and return it to the patient. Hollow, medical grade tubing facilities the fluid communication throughout the circuit, thus carrying the blood from the patient to and/or between each component, and returning it to the patient.

At least one vessel may be located within the circuit, with the vessel comprising an axial member having forward, medial and rearward portion, and defining a member lumen in fluid communication with the fluid circuit. The member lumen extends through the forward, medial and rearward portions, terminating at forward and rearward openings.

The member lumen defines a medial cylindrical inner surface within the member's medial portion, and forward and rearward cylindrical inner surfaces terminating at the forward and rearward openings. The inner diameter of the lumen in the medial portion is smaller than that of the forward and/or rearward portions. At least one gateway unitary with the member and defining a gateway lumen through the member's medial portion and in fluid communication with the member lumen is defined therein. The at least one gateway lumen terminates at a gateway opening at an outer end of the at least one gateway, with the gateway outer end operably engageable with an input/extraction conduit for fluid communication with the gateway lumen.

According to the well-known Venturi effect, a flow of blood through the smaller inner diameter of the member lumen within the medial portion results in a reduction in fluid pressure therein, thus resulting in the creation of a vacuum point within the member lumen of the medial portion such that a medication and/or fluid additive is drawn into the opening and lumen of the at least one gateway in fluid communication therewith. The reduced pressure within the medial portion also eliminates or minimizes blood loss through the at least one gateway.

The vessel defines a member outer surface having member forward and rearward engagement surfaces located proximal to both the respective forward and rearward ends. The forward and rearward engagement surfaces create a fluid-tight frictional connection with the inner surface of the tubing facilitating the fluid communication between circuit's components. The at least one gateway also defines a gateway outer surface having a gateway engagement surface located proximal to the gateway's outer end. The gateway engagement surface of the at least one gateway creates a fluid-tight connection with an input/extraction conduit utilized for infusing medications and/or fluids into the circuit or removing blood samples from the circuit.

An alternate embodiment of the access vessel further comprises at least one selectively occludable port unitary with the member and located forwardly or rearwardly of the medial portion, Unlike the aforementioned valved cap, the selectively occludable port allows for the insertion of a catheter or guide-wire into and through the vessel while preventing flow of blood therefrom. The at least one selectively occludable port defines an occludable port lumen through the member and in fluid communication with the member lumen. The at least one occludable port lumen terminates at an occludable port opening at an outer end of the at least one selectively occludable port, with the occludable port opening configured to accept an insertion of a catheter and/or guide-wire there-through for further insertion through the member lumen and ultimately into a patient.

An inwardly expandable tubular balloon is located within the at least one selectively occludable port, adjacent to an inner surface of the occludable port lumen to facilitate the port's selective occludability. In a deflated state, the volume of the tubular balloon is minimal such that an interior volume of the occludable port lumen remains undisturbed and un-occluded to allow for the flow of fluids or the insertion of catheters and/or guide-wires through the at least one occludable port. In an inflated state, the tubular balloon expands inwardly such that the interior volume of the occludable port lumen is occluded to block any fluid flow there-though. To facilitate an operation of the at least one selectively occludable port via the inflation and deflation of the tubular balloon, the balloon is in fluid communication with an actuatable gas or liquid source. When actuated, the gas or liquid source forces gas or liquid from the source and into the tubular balloon to cause the balloon to expand within the occludable port lumen.

In other embodiments, the at least one selectively occludable port may be removably connected to the vessel, with such connection including a connection to the outer end of the at least one gateway of the vessel. While this foregoing description and accompanying figures are illustrative of the present invention, other variations in system and method are possible without departing from the invention's spirit and scope.

### BRIEF DESCRIPTION OF THE DRAWINGS

FIG. 1A is a schematic diagram of an embodiment of the system;

FIG. 1B is a schematic diagram of another embodiment of the system;

FIG. 2 is a perspective view of an embodiment of the access vessel;

FIG. 3 is a sectional view of the embodiment of the access vessel of FIG. 2;

FIG. 4A shows a direct pressure measurement at various points of the circuit with an embodiment of the vessel defining a ⅜ inch inner diameter within its medial portion;

FIG. 4B shows a direct pressure measurement at various points of the circuit with an embodiment of the vessel defining a ¼ inch inner diameter within its medial portion;

FIG. 5A shows an air-fluid column differences at various points of the circuit with an embodiment of the vessel defining a ⅜ inch inner diameter within its medial portion;

FIG. 5B shows an air-fluid column differences at various points of the circuit with an embodiment of the vessel defining a ¼ inch inner diameter within its medial portion;

FIG. 6 shows an inward or outward fluid flow at various points of the circuit;

FIG. 7 is a bottom plan view of another embodiment of the access vessel;

FIG. 8 is a sectional view of the embodiment of the access vessel of FIG. 8;

FIG. 9 is a sectional view of the occludable port of FIG. 7 along plane A-A;

FIG. 10 is a sectional view of the occludable port of FIG. 7 along plane B-B;

FIG. 11A is a schematic view of an actuatable gas or liquid source and occludable port in an actuated state;

FIG. 11B is a schematic view of an actuatable gas or liquid source and occludable port in a non-actuated state;

FIG. 12 is a top plan view of one embodiment of a removably attachable occludable port showing hidden lines; and

FIG. 13 is a detailed perspective view showing hidden lines of the removable occludable port of FIG. 12.

### DESCRIPTION OF THE EMBODIMENTS

This invention relates to the infusion of medications and/or fluids into a patient, and/or the removal of blood samples therefrom. More specifically, this invention relates to an apparatus, system and method of administering such medication/fluid infusion into a patient and/or blood removal from a patient on extracorporeal membrane oxygenation (ECMO) and/or cardiopulmonary bypass (CPB) fluid circuits such that the need for utilizing central lines on the patient is avoided, thus also avoiding or minimizing the associated bloodstream infections and numerous other disadvantages associated with central line use and other means of vascular access. Additionally, this invention obviates any need for dialysis catheters as well. Finally, the invention permits for a safe and rapid access for endovascular procedures.

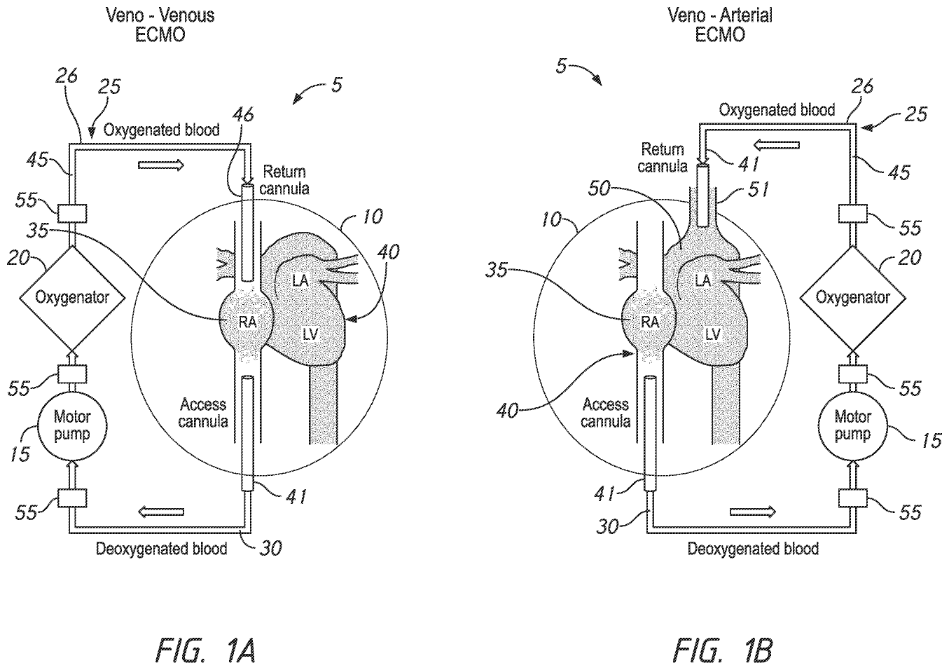

FIGS. 1A and 1B illustrate schematic diagrams of two representative embodiments out of all possible ECMO configurations of the system 5 for infusing medications and/or fluids, and/or removing blood samples from a patient 10. The system 5 comprises a pump 15 configured for fluid communication with the patient 10, and an oxygenator 20 in fluid communication with the pump and configured for fluid communication with the patient, with the pump, oxygenator and associated fluid communications comprising a fluid circuit 25. The fluid circuit 25 is utilized in a medical setting to receive the blood of the patient 10, oxygenate it, and return it to the patient. Hollow, medical grade tubing 26 facilities the fluid communication throughout the circuit 25, thus carrying the blood from the patient to and/or between each component, and returning it to the patient.

In the embodiment of FIG. 1A, known within the practice of medicine as a veno-venous ECMO circuit, the pump 15 creates a vacuum that draws deoxygenated blood 30 from the patient's right atrium “RA” 35 of the heart 40 and through an access cannula 41, thereafter pushing it through the oxygenator 20 under pressure, with such pressure further pushing the now oxygenated blood 45 from the oxygenator, through a return cannula 46, and back to the patient's right atrium. The access and return cannulas 41 and 46 are in fluid communication with the medical grade tubing 26 of the circuit 25.

In the embodiment of FIG. 1B, known as a veno-arterial ECMO circuit, the pump 15 creates a vacuum that draws deoxygenated blood 30 from the patient's right atrium 35 of the heart 40, thereafter pushing it through the oxygenator 20 under pressure, with such pressure further pushing the now oxygenated blood 45 from the oxygenator to a return cannula or graft 46, and to the patient's aorta 51.

Further variations of the circuit 25 are understood in the art, to comprise cardiopulmonary bypass (i.e., CPB) and other circuits as well. Unlike prior art ECMO, CPB and other similar fluid circuits, however, the present system 5 utilizes at least one access vessel 55 within with the fluid circuit 25. As illustrated within FIGS. 1A and 1B, the at least one vessel 55 may be located within the circuit 25 at various locations, to include: between the patient 10 and pump 15; between the pump 15 and oxygenator 20; or between the patient 10 and oxygenator 20. Vessel 55 can thus be applied to all ECMO configurations.

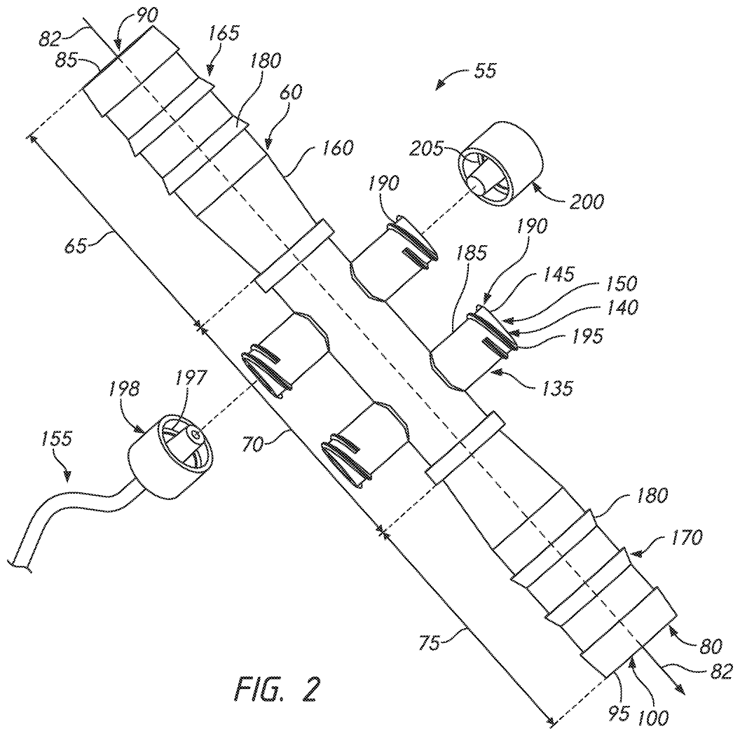

Referring to FIG. 2, in one embodiment, the vessel 55 comprises an axial member 60 having forward, medial and rearward portions 65, 70 and 75 and defining a member lumen 80 in fluid communication with the fluid circuit 25. The forward, medial and rearward portions 65, 70 and 75 are respectively upstream of one another with regard to fluid flow 82 (i.e., blood flow) though the member lumen 80. However, it is understood that the member is reversible such that the forward and rearward portions may be reversed about the medial portion without adversely affecting the vessel's functionality. The member lumen 80 extends through the forward, medial and rearward portions 65, 70 and 75 of the member 60, terminating at both a forward opening 85, defined in a forward end 90 of the forward portion, and a rearward opening 95 defined in a rearward end 100 of the rearward portion. The member lumen 80 within the forward and/or rearward portions of the member define an inner diameter of between about ¼-inches and ¾-inches, preferably between about ⅜-inches and ½-inches. Although not required, the inner diameter of the member lumen 80 with the forward and/or rearward portion may be about equal to the inner diameter of the tubing 26 of the circuit 25.

In a preferred embodiment of the invention, the vessel 55 comprises rigid, medical grade plastic such as polycarbonate, polypropylene, polyethylene and/or custom-made polymers, with all of the components of the vessel 55 being unitary with one another as a result of its underlying preferred manufacture via a precise plastic injection molding process. The rigidity of the plastic allows the vessel 55 to operably engage the more flexible plastic tubing 26 of the circuit 25 that facilitates the fluid communication between the pump 15, oxygenator 20 and patient 10. It is understood, however, that the vessel 55 and all or some of its underlying components may comprise polycarbonate, aluminum, stainless steel as well as aluminum and other similar materials known in the art for providing the desired material properties.

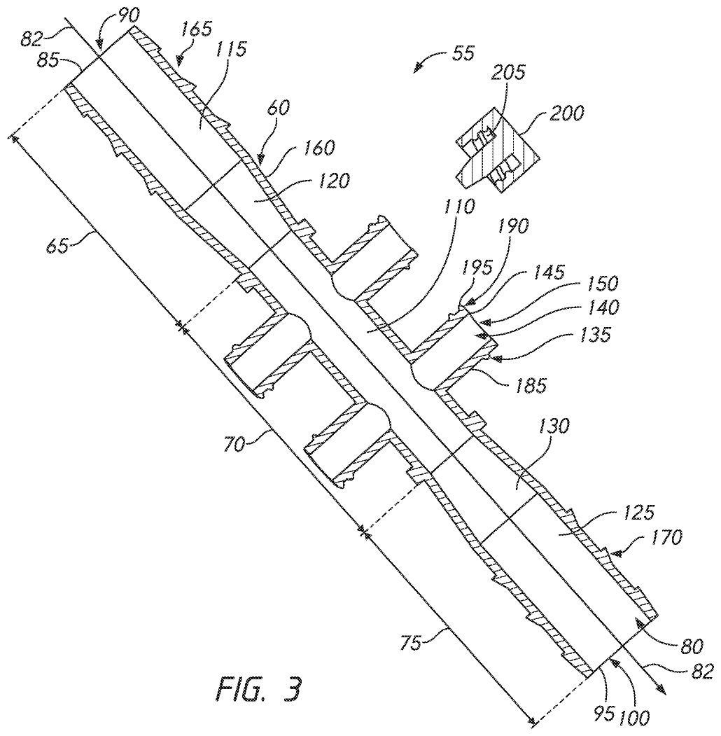

As better illustrated in the sectional view of FIG. 3, the member lumen 80 defines a medial cylindrical inner surface 110 within the member's medial portion 70. Within the member's forward portion 65, the member lumen 80 defines a forward cylindrical inner surface 115 at the forward opening 85 and having a diameter larger than that of the medial cylindrical inner surface 110. Located rearwardly of the forward cylindrical inner surface 115, the member lumen further defines a forward frusto-conical transition inner surface 120 within the member's forward portion 65 that transitions to the smaller diameter of the medial inner cylindrical surface 110 of the member's medial portion 70. Similarly, the within member's rearward portion 75, the member lumen 80 defines a rearward cylindrical inner surface 125 at the rearward opening 95, and also having a diameter larger than that of the medial cylindrical inner surface 110. Located forwardly of the rearward cylindrical inner surface 125, the member lumen 80 defines a rearward frusto-conical transition inner surface 130 within the member's rearward portion 75 that transitions to the smaller diameter of the medial inner cylindrical 110 surface of the member's medial portion 70.

Referring now to both FIGS. 2 and 3, the vessel 55 further comprises at least one gateway 135 unitary with the member 60 and defining a gateway lumen 140 into the member's medial portion 70 and in fluid communication with the member lumen 80 defined therein. The gateway lumen 140 defines an inner diameter of between about 1 mm and about 10 mm, preferably between about 2 mm and 6 mm, and optimally about 4 mm. Although FIGS. 2 and 3 illustrate four such gateways 135, it is understood that the vessel 55 may comprise any number of one or more gateways. The at least one gateway lumen 140, in turn, terminates at a gateway opening 145 at an outer end 150 of the at least one gateway 135, with the gateway outer end operably engageable with an input/extraction conduit 155 for fluid communication with the gateway lumen 140. Furthermore, multiple gateways of the at least one gateway 135 may be located opposite of one another, staggered on the same plane, staggered on a spiraling plane etc.

According to the well-known Venturi effect, a flow of blood through the smaller inner diameter of the member lumen 80 within the medial portion 70 results in a reduction in fluid pressure therein, thus resulting in the creation of a vacuum point within the member lumen of the medial portion such that a medication and/or fluid additive is drawn into the opening 145 and lumen 140 of the at least one gateway 135 in fluid communication therewith. The reduced pressure within the medial portion 70 also eliminates or minimizes blood loss through the at least one gateway 135. In one embodiment of the invention, the precise volumetric flow rate of the medication and/or fluid into and through the gateway is achieved via a control of the cross-sectional area (i.e., diameter) of the member lumen 80 within the member's medial portion 70, The relationship between the components of the vessel 55, the blood flowing there-through, and the medication and/or other fluids that is drawn to the at least one gateway 135, and/or the blood flow prevented from exiting the at least one gateway, is readily ascertained via the Bernoulli equation:

P₁+(½)ρ₁υ₁+ρ₁g₁h₁==P₂+(½)ρ₂υ₂²+ρ₂g₂h₂
P₁=pressure of blood within the forward portion 65 of the member 60;
ρ₁=density of blood flog flowing through the forward portion 65 of the member 60;
υ₁=velocity of blood flow through the forward portion 65 of the member 60 (υ₁=Q₁/(πR₁²));
Q₁=flow rate of blood through the forward portion 65 of the member 60;
R₁=internal radius of the lumen 80 within the forward portion 65 of the member 60 (R₁=½ diameter D₁);
g₁=acceleration due to gravity existing within the forward portion 65 of the member 60;
h₁=height from the ground of the forward portion 65 of the member 60;
P₂=pressure of blood within the medial portion 70 of the member 60, located downstream of the forward portion 65;
ρ₂=density of blood flowing through medial portion 70 of the member 60, located downstream of the forward portion 65;
υ₂=velocity of blood flow through the median portion 70 of the member 60 (υ₂=Q₂/(πR₂²)), located downstream of the forward portion 65;
Q₂=flow rate of blood through the medial portion 70 of the member 60, located downstream of the forward portion 65;
R₂=internal radius of the medial portion 70 of the member 60 (R₂=½ diameter D₂), located downstream of the forward portion 65;
g₂=acceleration due to gravity within the medial portion 70 of the member 60, located downstream of the forward portion 65; and
h₂=height from the ground of the medial 70 portion of the member 60, located downstream of the forward portion 65.

Given the known value for patient blood pressure (P₁) existing within the forward portion 65 of the member 60, we seek to determine the downstream pressure (P₂) of the blood existing within the member's medial portion 70. Assuming equivalent values existing throughout the member 60 for blood density (ρ1=ρ2), the acceleration due to gravity (g₁=g₂), and the height from the ground (h₁=h₂), these variables cancel each other out such that P₂ may be determined as follows:

P₂=(P₁+(½)ρ₁υ₁²)−(½)ρ₂υ₂², where υ₁=Q₁/(πR₁²) and υ₂=Q₂/(πR₂²).

We know, via clinical determination, the pressure (P₁) of the blood existing within the forward portion 65 of the member 60 (i.e., between about 15 and 300 mm Hg, optimally about 240 mm Hg). We also know the flow rate (Q₁) of the blood though the forward portion 65 of the member 60 (i.e., between about 3 and 5 L/min, optimally about 4 L/min), and that this flow rate remains constant (i.e., Q₁=Q₂) through the medial portion 70 of the member, regardless of the internal diameter of the member's lumen 80, Similarly, we additionally know the density (ρ₁) of the blood (i.e. about 1 gm/ml) through the forward portion 65 of the member 60, and that this density also remains constant (i.e., ρ₁=ρ₂) through the medial portion 70 of the member, also regardless of the internal diameter of the member's lumen 80. Finally, we further know the radius (R₁)=½ the diameter (D₁; i.e. standardized circuit tubing 26 having an internal lumen diameter of about ⅜-inch and about ½-inch, respectively) of the forward portion 65.

Theoretically, we can thus vary the radius (R_{2;}=½ the diameter of D₂) of the medial portion 70 to determine to determine numerous optimal values for the pressure (P2) existing within the medial portion of the at least one vessel 55. Thus, assuming for the sake of example: P₁=about 250 mm Hg; Q₁=Q₂=about 4 L/min=about 4000 ml/min; ρ₁=ρ₂=about 1 gm/ml; D₁=about 0.375 in=about 0.9525 cm=about 9.525 mm for venous tubing; and D₂=about 0.283 in=about 0.7189 cm=about 7.189 mm, we can calculate P₂ to be about 243 mm Hg; which represents an approximate 25% reduction in lumen diameter from the forward portion 65 of the member to the medial portion 70. Similarly, utilizing an approximate 50% reduction in diameter from the forward portion 65 to the medial portion 70, namely, where D₁=about 0.375 in and D₂=about 0.1875 in, we calculate P₂ to be about 200 mm Hg.

To verify the foregoing, tests of the vessel 55 within a closed-loop system, comprising of all components of the fluid circuit 25 of FIGS. 1A and 1B, were conducted. In place of a patient 10, the inflow and outflow tubing 26 was instead connected to one another using an adapter (not shown) to create the closed-loop system. Water was utilized in place of blood due to the fact that blood and water possess about the same fluid density. The Venturi effect at the medial portion 70 of the vessel 55 was noted using three different measurement techniques at water flows of 4 L/min through the circuit 25.

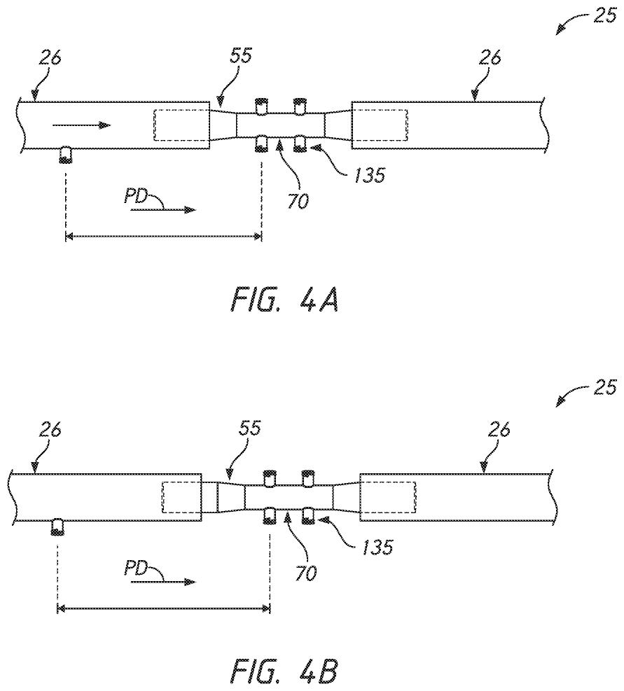

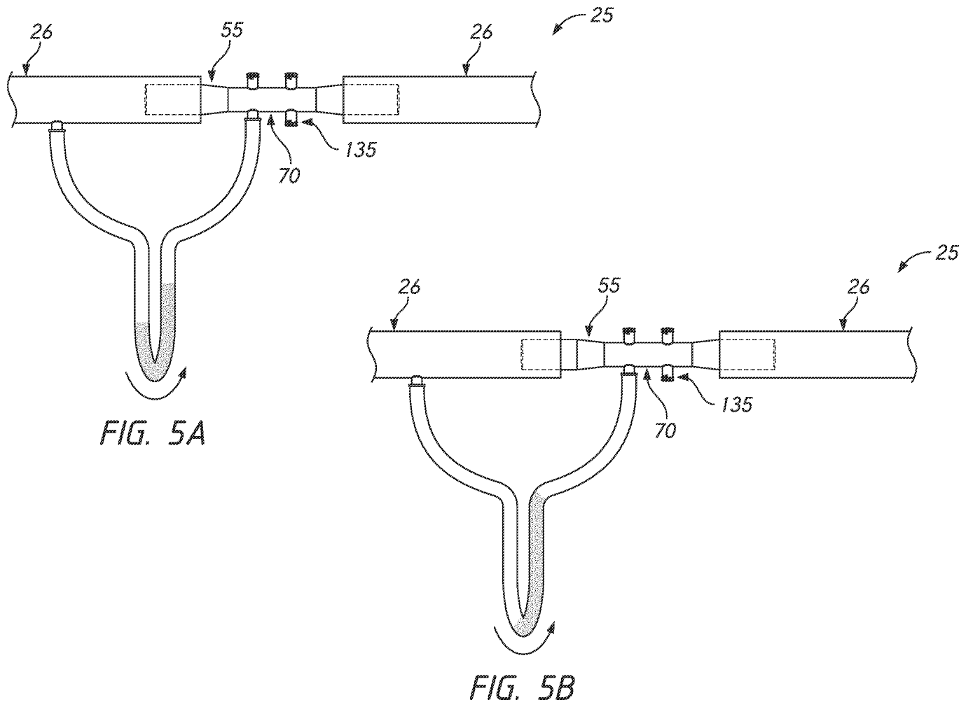

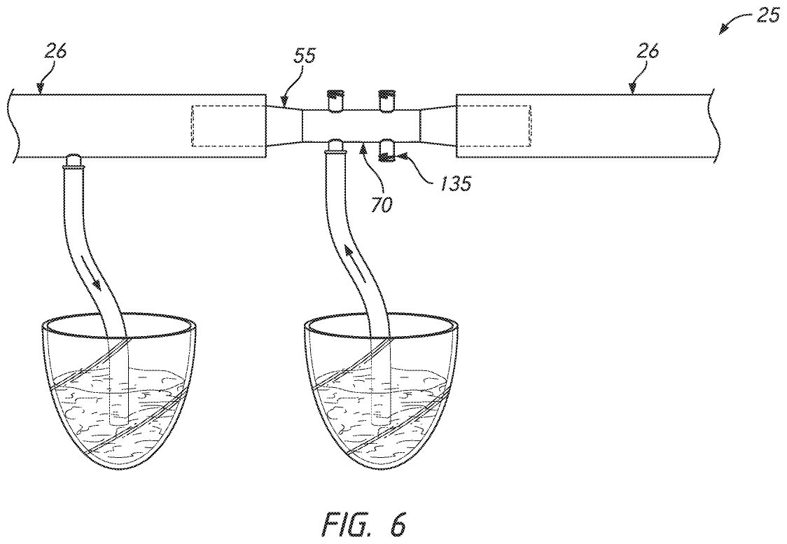

First, upon measuring the fluid pressures at various points in the circuit 25, a pressure differential “PD” between a ½-inch inner diameter tubing 26 and the medial portion 70 of the vessel 55 was observed to be directly related to the vessel's medial portion inner diameter, namely, a 7 mm Hg drop PD for a ⅜-inch medial portion inner diameter (FIG. 4A) and a 42 mm Hg drop PD for a ¼-inch medial portion inner diameter (FIG. 4B). Next, a manometer was connected to demonstrate the existence of the pressure differential PD existing between ½-inch inner diameter tubing 26 and the medial portion 70 inner diameters of the vessel 55. For either a ⅜-inch (FIG. 5A) or ¼-inch (FIG. 5B) medial portion 70 inner diameter, the water column was significantly displaced towards the diameter of the vessel's medial portion; again indicating the existence of the pressure differential PD between a ½-inch inner diameter tubing and the vessel's medial portion inner diameter, as well as a vacuum effect occurring within the medial portion itself. Finally, the ½-inch inner diameter tubing 26 and the vessel's medial portion 70 were simultaneously connected to containers filled with dyed-water (FIG. 6). For both the ⅜-inch and the ¼-inch medial portion 70 inner diameters, the dyed-water was drawn into the vessel's medial portion whereas water was nonetheless evacuated from the circuit 25 at the level of the ½-inch inner diameter tubing 26; again demonstrating a vacuum effect at the vessel's medial portion with a positive outwards pressure concurrently exerted at the level of the ½-inch inner diameter tubing.

For the sake of completeness, the foregoing pressure differential tests were also conducted while utilizing a one-way valve adapter (to be further discussed) to occlude the at least one gateway 135 of the vessel 55. Such valve adapters are typically utilized in drawing blood samples from a patient, via the attachment of a syringe there-through, while preventing an outflow of blood there-from in the absence of the attached syringe. Despite the vacuum present within the medial portion 70 of the vessel 55, no external air was drawn into the circuit 25 through the valve adapter. As such, the access vessel 55 is safe for use with valve adapters commonly used in facilitating blood draws/sampling from the fluid circuit 25.

The foregoing calculated pressures (P₂) and associated diameters (D₂) are critical because they ensure fluid pressure reductions that eliminate or minimize blood loss through the at least one gateway 135 and/or sufficient to draw medications/fluid into the at least one gateway, while also not increasing the blood velocity within the vessel's medial portion 70 to dangerous levels that may result in blood cell sheer (leading to hemolysis, for example). They further ensure the presence the desired vacuum within the vessel's medial portion 70 sufficient to draw medicine and/or fluid into the circuit 25, yet insufficient to draw undesirable air through a valve cap (to be further discussed) occluding the at least one gateway 135. Nonetheless, although about 25% and 50% diameter reductions between tubing and medial portion inner diameters are recited by example herein, other diameters (D₂) are nonetheless possible as well, to include those resulting in reductions of between about 1% and 99%.

Referring again to FIG. 2, the vessel defines a member outer surface 160 having member forward and rearward engagement surfaces 165 and 170 located proximal to both the respective forward and rearward ends 90 and 100 of the member's respective forward and rearward portions 65 and 75, The forward and rearward engagement surfaces 165 and 170 create a fluid-tight frictional connection with the inner surface 175 (not shown) of the tubing 26 facilitating the fluid communication between circuit's components. In the embodiment illustrated in FIG. 2, the respective engagement surfaces comprise a plurality of inwardly angled ridges 180 encircling the member's outer surface 160. However, it is understood that the forward and rearward engagement surfaces 165 and 170 may comprise helical threads and other structures, such as common Luer locks, for engagement with like connectors attached or defined at the ends of the tubing 26.

The at least one gateway 135 also defines a gateway outer surface 185 having a gateway engagement surface 190 located proximal to the gateway's outer end 150. The gateway engagement surface 190 of the at least one gateway 135 creates a fluid-tight connection with the input/extraction conduit 155. In the embodiment illustrated in FIG. 2, the gateway engagement surface 190 comprises an external helical thread 195, commercially known as a Luer Lock coupling, fir threaded engagement with an internal thread 197 of a like coupling 198 defined on an end of the input/extraction conduit 155. However, it is understood that the gateway engagement surface 190 of the at least one gateway 135 may comprise a plurality of inwardly angled ridges, and other similar structure surfaces understood in the art as well as creating a fluid-tight fit.

With regard to infusing medications and/or fluids, and/or removing of blood samples through the at least one gateway 135, utilization of the Luer Lock is preferred to allow for the standardized connection of the access vessel 55 to various embodiments of the input/extraction conduit 155, to comprise, without limitation, connection to: a syringe for quickly injecting larger volumes of medication and/or fluid into a patient during critical care scenarios; common IV drip containers for slowly infusing pre-determined volumes of such medicine and/or fluids into the patient; and/or Patient-Controlled Analgesia (i.e., PCA) pumps for administering precise units of patient-controlled pain medications, as prescribed by a qualified medical professional; and/or utilization in conjunction with a hemodialysis (HD) machine or continuous renal replacement therapy (CRRT) in cases of renal failure. As discussed earlier, current techniques to perform HD or CRRT on ECMO patients rely on the placement of peripherally inserted central dialysis catheters, which not only have the same downsides of central IV lines, but they are highly prone to complete obstruction/occlusion requiring anti-coagulants or catheter replacement. This issue occurs due to the higher resistance of the dialysis catheter's small caliber. However, the current embodiment allows the at least one gateway 135 to directly connect to a much larger flow channel thus preventing any impediments to the HD/CRRT flow.

For embodiments of the at least one vessel 55 having more, than one gateway of the at least one gateway 135, the foregoing input/extraction conduits may be connected to the respective multiple gateways for simultaneous or successive injection and/or infusion of the respective medications and/or fluids into a patient. For example, if a patient incurs a sudden drop in blood pressure through internal bleeding) while the infusion pump is infusing medications through one gateway of the vessel, a medical practitioner can rapidly inject large volumes of pressure balancing fluids, such as blood or saline, through another gateway of the vessel. Similarly, simultaneous blood sampling can also be performed.

In the vessel 55 embodiments of FIGS. 2 and 3, at least one cap 200 is preferably used in association with the at least one gateway 135 of the vessel 55 to close the gateway when not in use when not connected to an input/extraction conduit 155). The cap 200 preferably is configured for operable engagement with the gateway engagement surface 190 to both frictionally engage and seal the at least one gateway 135. In a preferred embodiment, the cap 200 defines an internal helical thread 205 that engage the external thread 195 of the at least one gateway 135; with such threads preferably comprising the standardized Luer Lock configuration. Although the cap 200 illustrated herein comprises a standard occluding cap that prevents fluid flow entirely through the at least one gateway 135, a valved cap (not shown), that only allows for flow with external suction or vacuum, may be utilized as well. These valved caps prevent flow when not engaged, and as such, may be unitary with the access vessel 55. More specifically, the valved caps facilitate the removal of blood from the circuit 25 via the attachment of the input extraction conduit 155 comprising a common syringe, or that relating to a vacuum tube (i.e., Vacutainer), while preventing an outflow of blood there-from in the absence of the attached syringe.

Again, for embodiments of the at least one vessel 55 having more than one gateway of the at least one gateway 135, the foregoing input/extraction conduits may be connected to the respective multiple gateways for simultaneous or successive removal/sampling of blood from the patient. Similarly, input/extraction conduits may be connected to one or e gateways for simultaneous or successive injection and/or infusion of the respective medications into the patient while other input/extraction conduits may be connected to the remaining one or more gateways for simultaneous or successive removal/sampling of blood from the patient.

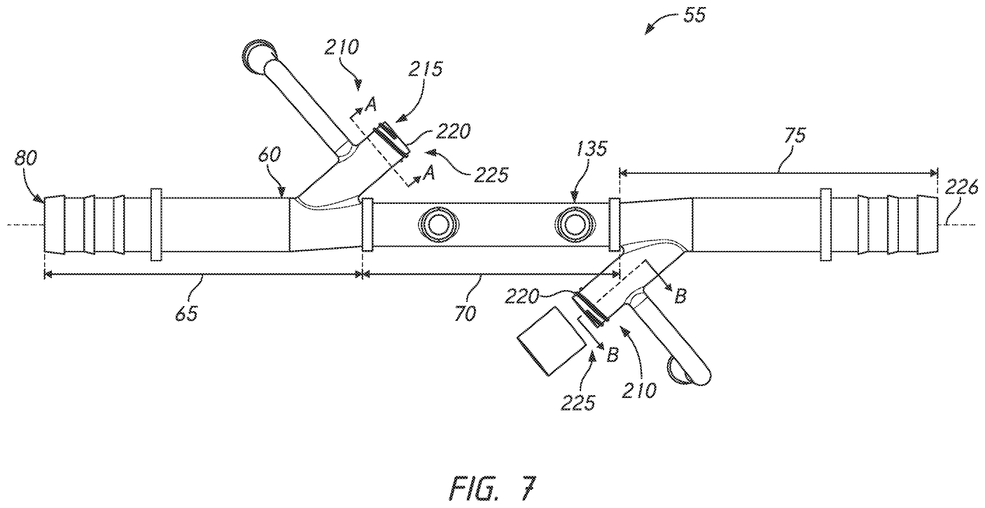

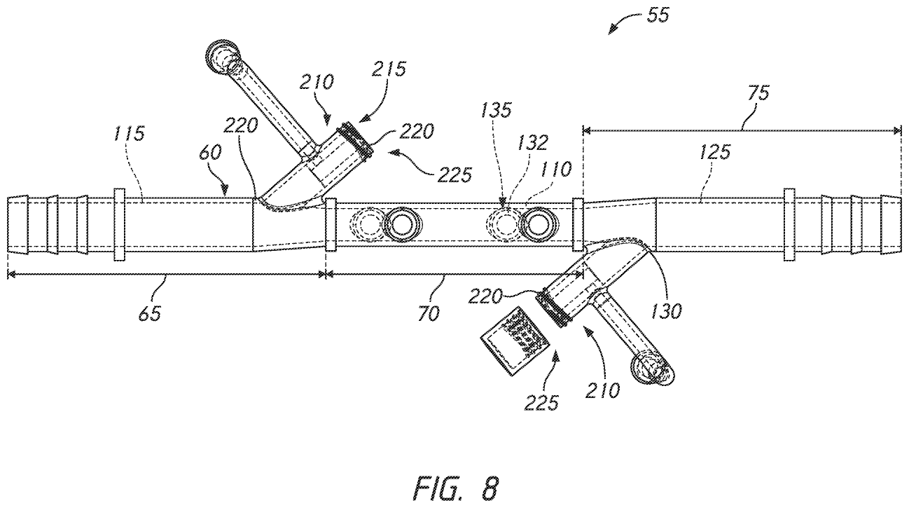

Referring to FIGS. 7 and 8, an alternate embodiment of the access vessel 55 further comprises at least one selectively occludable port 210 unitary with the member 60 and located forwardly or rearwardly of the medial portion 70. Unlike the aforementioned valved cap, the selectively occludable port allows for the insertion of a catheter or guide-wire into and through the vessel 55 while preventing the outward flow of blood therefrom. The at least one selectively, occludable port 210 defines an occludable port lumen 215 into the member and in fluid communication with the member lumen 80. The selectively occludable port lumen 210 defines an inner diameter of between about 1 mm and about 15 mm, preferably between about 5 mm and 9 mm, and optimally about 7 mm. Although FIG. 7 illustrates two such selectively occludable ports 210 located in apposition of one another about the medial portion 70, it is understood that the vessel 55 may comprise any number of one or more selectively occludable ports. Furthermore, although FIGS. 7 and 8 show each of the at least one selectively occludable ports 210 defining their respective occludable port lumens 215 through the respective forward and rearward frusto-conical transition inner surfaces 120 and 130 of the member lumen 80, it is understood that the at least one occludable port lumen may be defined through the forward or rearward cylindrical inner surfaces 115 and 125 (FIG. 8) as well.

The at least one occludable port lumen 215, in turn, terminates at an occludable port opening 220 at an outer end 225 of the at least one selectively occludable port 210, with the occludable port opening configured to accept an insertion of a catheter and/or guide-wire there-through for further insertion through the member lumen 80 and ultimately into a patient. To facilitate an ease of insertion of the catheter and/or guide-wire through the at least one selectively occludable port 210, into and through the member lumen 80 and ultimately into a patient, the at least one selectively occludable port is oriented at an angle in relation to an axis 226 of the member lumen 80 such that any catheter and/or guide-wire inserted through the at least one occludable port is directed ultimately into the larger-diameter cylindrical inner surfaces 115 or 125 of the respective forward or rearward portions 65 or 75. The angle of the at least selectively one occludable port 210 from the axis 226 is between about 1 degree and 179 degrees, preferably between about 25 degrees and 165 degrees, and optimally about 45 degrees from the axis 226. Directing the catheter and/or guide-wire into the smaller-diameter inner cylindrical surface 110 of the medial portion 70 is to be avoided due to the likely constriction of the medial portion about the catheter and/or guide-wire, and also because of the likelihood of the catheter and/or guide-wire's forward end catching and snagging on the interior opening 132 of the at least one gateway 135 terminating within the medial cylindrical inner surface. Although FIGS. 7 and 8 illustrate the at least one selectively occludable port 210 oriented at about a 45 degree angle from an axis of the member lumen 80, it is understood that the at least one occludable port may be oriented at other angles as well, to include those recited herein.

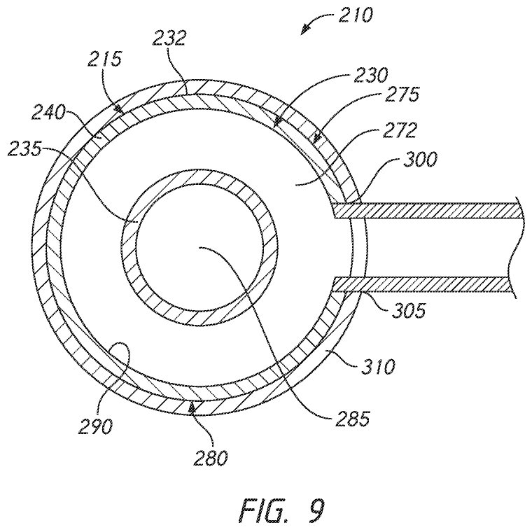

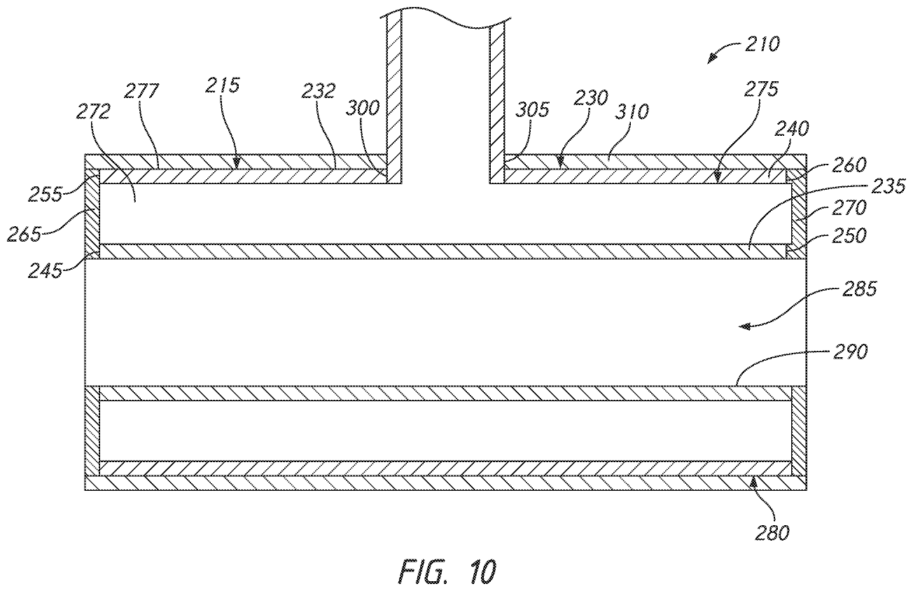

Referring to FIGS. 9 and 10, which illustrate sectional views of the at least one occludable port 210 of FIG. 7 along respective planes A-A and B-B, an inwardly expandable tubular sleeve 230 located within the at least one selectively occludable port 210, adjacent to an inner surface 232 of the occludable port lumen 215 to facilitate the port's selective occudability. In one embodiment, the expandable tubular sleeve 230 comprises inner and outer tubular sheets 235 and 240 having forward ends 245 and 255 and rearward ends 250 and 260 (FIG. 10) terminating at respective approximate forward and rearward end-rings 265 and 270 to create an internal closed volume 272 of fluid-tight, inflatable, tubular balloon 275. Although FIG. 10 illustrates end rings 265 and 270 as displacing the outer sheet ends 255 and 260 vertically from the inner sheet ends 245 and 250, is understood that no vertical displacement is created by the rings of the balloon 275 such that the upper sheet ends and lower sheet ends co-terminate adjacent with one another.

An outer surface 277 of the outer sheet 240 is located adjacent, and optionally secured to, the inner surface 232 of the occludable port lumen 215. The optional securement of the outer sheet 240 to the inner surface 232 can comprise an adhesive, thermal or other bonding means understood in the art as bonding two surfaces to one another. In a deflated state, closed volume 272 of the tubular balloon 275 is minimal such that the inner sheet 235 is located adjacent to the outer sheet 240 and an interior volume 280 of the occludable port lumen 215 remains undisturbed and un-occluded to allow for the flow of fluids or the insertion of catheters and/or guide-wires through the at least one occludable port 210. In the absence of a catheter and/or guide-wire located within the balloon's center 285, the interference fit of the balloon's inner sheet 235 with itself will minimize or “close” the balloon's center such that the flow of blood outwardly there-though is minimized or occluded.

In an inflated state, the inner tubular sheet 235 inwardly separates from the outer tubular sheet 240, as the internal closed volume 272 of the tubular balloon 275 fills with a gas or liquid, and expands inwardly to create an interference fit with itself such that the interior volume 280 of the occludable port lumen 215 is occluded to block any fluid flow there-though. However, because the inner tubular sheet 235, despite creating an interference fit with itself, nonetheless continues to define a tubular center 285 there-through, catheters and/or guide-wires medical devices may be inserted through the center, or otherwise remain located within that center, while the tubular balloon 275 remains inflated.

Thus, when inflated, an interference fit exists between an inner surface 290 of the inner sheet 235 of the balloon and an outer surface of the catheter or guide-wire to occlude any fluids from otherwise flowing between these surfaces and through the occludable port lumen 215 about the catheter or guide-wire. To aid in the any insertion or retraction of the catheter or guide-wire into or out of the inflatable balloon's center 285 of the inflated tubular balloon 275, the inner surface 290 of the inner tubular sheet 235 is optionally coated with a hydrophilic coating to reduce any frictional resistance between the inner sheet and the catheter or guide-wire located within the balloon's center.

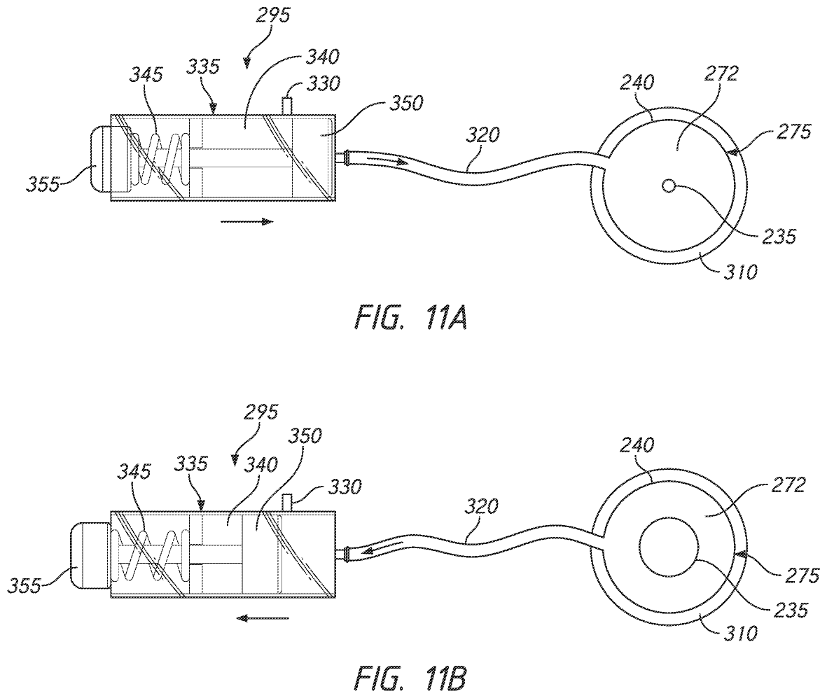

To facilitate an operation of the at least one selectively occludable port 210 via the inflation and deflation of the tubular balloon 275, the balloon's closed volume 272 is in fluid communication with an actuatable gas or liquid source 295. In the embodiment of FIG. 11, respective openings 300 and 305 are defined through both the outer sheet 240 of the tubular balloon 275 and a surrounding wall 310 of the occludable port 210. An inner end 315 of a source tube 320 is sealingly connected about the opening 300 of the tubular outer sheet 240, with the source tube extending though the opening 305 of the occludable port wall 310. An outer end 325 of the source tube 320 is sealingly connected to the actuatable gas or liquid source 295, with the source preferably biased in a de-actuated state to keep the tubular balloon 275 deflated.

When actuated (FIG. 11A), the gas or liquid source 295 forces gas or liquid from the source, through the source tube 320 and into the internal closed volume 272 of the tubular balloon 275 to cause the balloon to expand within the occludable port lumen 215. A lock or stay 330 is optionally included with the actuatable gas or liquid source 295 such that a triggering of the lock or stay causes the liquid source to remain actuated, and the tubular balloon 275 inflated, until the lock or stay is de-triggered to allow the source to return its biased de-actuated state. In one embodiment, the actuatable gas or liquid source 295 comprises a spring-biased, button-actuated closed piston and cylinder arrangement 335 storing the gas or liquid within the cylinder 340. The spring 345 biases piston 350 rearwardly within the cylinder 340 such that a pressing of the button 355 will counteract the bias of the spring 345 to force the piston forwardly within the cylinder; thus forcing the gas or liquid from the cylinder, through the source tube 320, and into the tubular balloon 275; thus causing the balloon to inflate.

Of course, when the button 355 is released (FIG. 11B), the spring 345 causes the piston 350 to retract from the cylinder 340, thus drawing the gas or liquid from the tubular balloon 275 and into the cylinder via the source tube 320, thus causing balloon to again deflate. Although a button-actuated piston and cylinder arrangement 335 is illustrated and disclosed herein, it is understood that other arrangements understood in the art are nonetheless possible. Also, although the actuatable source 295 is biased in the de-actuated position to bias the tubular balloon 275 in a deflated state, it is understood that the source may be biased in the actuated position to bias the balloon in an inflated state as well.

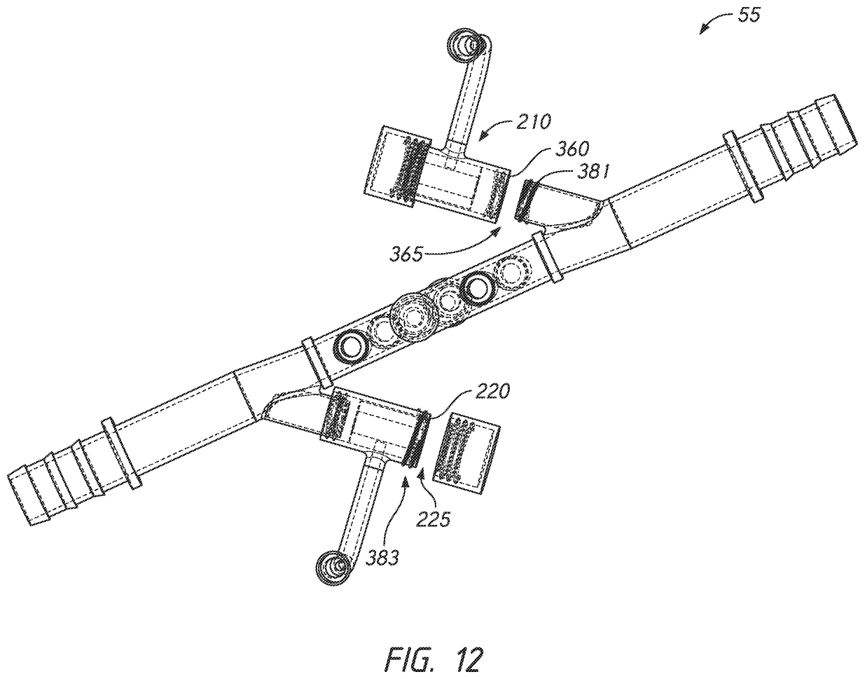

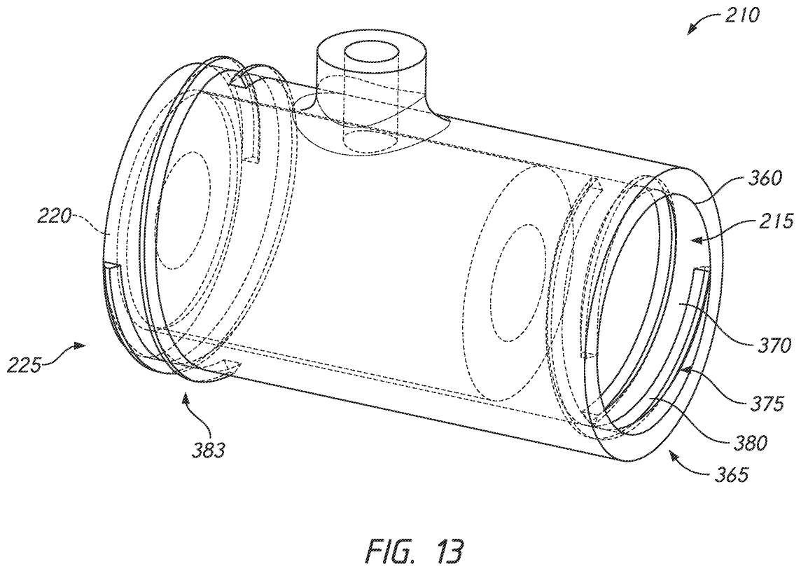

Although FIGS. 7 and 8 illustrate the at least one selectively occludable port 210 and associated components as being unitary with the access vessel 55, it is understood that the at least one selectively occludable port may be removably connected to the vessel as well. Thus, as illustrated in the embodiment of FIGS. 12 and 13, the occludable port lumen 215 of the at least one removably connected, selectively occludable port 210 terminates at an occludable port inner opening 360 located at an inner end 365 of the at least one occludable port 210 located opposite the outer opening 220 located at the port's outer end 225. The at least one occludable port 210 also defines a port inner surface 370 having a port engagement surface 375 respectively located proximal to at least the port's inner end 365 (FIG. 13). The port engagement surface 375 of the at least one selectively occludable port 210 creates a fluid-tight connection with the access vessel member 55.

In the embodiment illustrated in FIGS. 12 and 13, the occludable port engagement surface 375 comprises an inner helical thread 380, again, commercially known as a Luer Lock coupling, for threaded engagement with the outer thread 381 of a like coupling (FIG. 12) defined on the member 60 of the access vessel 55. However, it is understood that the occludable port engagement surface 375 of the at least one selectively occludable port 210 may comprise a plurality of inwardly angled ridges, and other similar structure surfaces understood in the art as well as creating a fluid-tight fit. It is further understood that the outer end 225 of the at least one occludable port 210 may also define and an engagement surface 383 as well such that engagement surfaces are located at each end of the port. In further embodiments, the at least one removably connected, selectively occludable port 210 defines a port lumen 215 inner diameter and outer connectable surfaces to facilitate a connection to the outer end of the at least one gateway 135 of the vessel 55. In addition to balloon occludability while obtaining access, an end cap with a central opening of varying diameters that correspond to the wire and/or catheter diameters being introduced can be threaded onto the balloon occludable port to provide further protection against blood leakage.

In use for infusing medications and/or fluids into a patient, a pump configured for fluid communication with a patient is provided. An oxygenator in fluid communication with the pump, and configured for fluid communication with a patient, is also provided. The pump, oxygenator and associated fluid communications comprise a fluid circuit. At least one access vessel is located within the fluid circuit, with the vessel comprising an axial member having forward, medial and rearward portions and defining a member lumen in fluid communication with the fluid circuit. The member lumen extends through the forward, medial and rearward portions of the member and defines a forward opening in the forward portion and a rearward opening in the rearward portion.

The lumen further defines an inside diameter within the medial portion that is smaller than an inside diameter defined within both the forward and rearward portions of the member. The vessel further comprises at least one gateway defining a gateway lumen through the medial portion of the member and in fluid communication with the member lumen. The gateway lumen further defines a gateway opening at an outer end of the gateway, with the gateway outer end operably engageable with an input/extraction conduit for fluid communication with the gateway lumen.

The at least one gateway of the vessel is utilized for infusing medications or other fluids into the fluid circuit, or for extracting blood samples from the circuit. An input/extraction conduit is connected to the at least one gateway for infusing medications or fluids into the circuit or extracting blood samples from the circuit.

When infusing medications or other fluids into the circuit, the input/extraction conduit may comprise, without limitation, connection to: a syringe for quickly injecting larger volumes of medication and/or fluid into patient during critical care scenarios; common IV drip containers for slowly infusing pre-determined volumes of such medicine and/or fluids into the patient; and/or Patient-Controlled Analgesia (i.e., PCA) pumps for administering precise units of patient-controlled pain medications, as prescribed by a qualified medical professional; and/or utilization in conjunction with a hemodialysis (HD) machine or continuous renal replacement therapy (CRRT) in cases of renal failure.

When extracting blood samples from the circuit, the input/extraction conduit may comprise a common syringe, or that relating to a vacuum gibe (i.e., Vacutainer). Occlusion caps and/or valved caps may be attached to the at least one gateway to facilitate the selective use of any one or more of the gateways simultaneously sequentially.

In use in another embodiment, the at least one access vessel is located within the fluid circuit, with the vessel further comprising at least one selectively occludable port defining a port lumen through the member and in fluid communication with the member lumen, and an inflatable tubular balloon defined within the port lumen. The port lumen further defines a port opening at an outer end of the port, with the tubular balloon selectively inflatable to selectively occlude the port lumen. A small amount of an actuatable gas or liquid source is pushed to inflate the tubular balloon to occlude the port lumen, and released to deflate the tubular balloon to open the port lumen. In use in yet another embodiment, the at least one selectively occludable port is removably connected to the member.

While this foregoing description and accompanying figures are illustrative of the present invention, other variations in system and method are possible without departing from the invention's spirit and scope.

We claim:

1. An access vessel for use in a fluid circuit having at least one pump configured for fluid communication with a patient, and at least one oxygenator in fluid communication with the at least one pump and configured for fluid communication with the patient, the access vessel comprising:
an axial member defining forward, medial and rearward portions and an interior member lumen, said member lumen extending through the forward, medial and rearward portions of the member and defining a forward opening at a forward end of the forward portion and a rearward opening at a rearward end of the rearward portion;
an outer surface defined by the member, said outer surface defining a forward engagement surface at the forward end and a rearward engagement surface that the rearward end, the forward and rearward engagement surfaces each configured for fluid-tight engagement with the fluid circuit for fluid communication with the member lumen, the member lumen defining a medial cylindrical inner surface within the medial portion having a diameter that is smaller than at least a diameter of a forward inner cylindrical surface defined within the forward portion;
at least one gateway defining a gateway lumen through the medial portion of the member in fluid communication with the member lumen, said gateway lumen further defining a gateway opening at an outer end of the gateway, said gateway outer end operably engageable with an input conduit for fluid communication with the gateway lumen.

2. The access vessel of claim 1 wherein the medial cylindrical inner surface within the medial portion is smaller than a diameter of a rearward inner cylindrical surface defined within the rearward portion, the member lumen further defining a forward frusto-conical transition surface located rearwardly of the forward cylindrical inner surface that transitions to the smaller diameter of the medial inner cylindrical surface of the member's medial portion, the member lumen further defining a rearward frusto-conical transition surface located forwardly of the rearward cylindrical inner surface that transitions to the smaller diameter of the medial inner cylindrical surface of the member's medial portion.

3. The access vessel of claim 2 wherein the at least one input conduit is in removable fluid communication with a fluid source via a removable connection located there-between, the fluid source selected from a group consisting of a syringe, an IV drip container, and an infusion pump.

4. The access vessel of claim 2 wherein an outward flow of fluid through the at least one input conduit is prevented by an occlusion device via a removable connection located there-between, the occlusion device selected from a group consisting of an occlusion cap and an inwardly-biased one-way valve.

5. The access vessel of claim 3 or 4 wherein the removable connection comprises a Luer lock.

6. The access vessel of claim 2 wherein a fluid flow rate through the member lumen is about equal to a fluid flow rate generated by the at least one pump.

7. The access vessel of claim 6 wherein the reduction in diameter of the medial cylindrical inner surface from the forward cylindrical inner surface defines a percentage reduction selected from a group consisting of about 25 percent and about 50 percent.

8. The access vessel of claim 2 further comprising at least one selectively occludable port defining a port lumen through the member and in fluid communication with the member lumen, and an inflatable tubular balloon defined within the port lumen, said port lumen further defining a port opening at an outer end of the port, and said tubular balloon selectively inflatable to selectively occlude the port lumen.

9. The access vessel of claim 8 wherein the at least one selectively occludable port is removably connected to the member.

10. An improved fluid circuit of the type having at least one pump configured for fluid communication with a patient, and at least one oxygenator in fluid communication with the at least one pump and configured for fluid communication with the patient, the improvement comprising:
an access vessel in fluid communication with the circuit, the access vessel comprising an axial member defining forward, medial and rearward portions and an interior member lumen, said member lumen extending through the forward, medial and rearward portions of the member and defining a forward opening at a forward end of the forward portion and a rearward opening at a rearward end of the rearward portion;
an outer surface defined by the member, said outer surface defining a forward engagement surface at the forward end and a rearward engagement surface that the rearward end, the forward and rearward engagement surfaces each configured for fluid-tight engagement with the fluid circuit for fluid communication with the member lumen, the member lumen defining a medial cylindrical inner surface within the medial portion having a diameter that is smaller than at least a diameter of a forward inner cylindrical surface defined within the forward portion;
at least one gateway defining a gateway lumen through the medial portion of the member in fluid communication with the member lumen, said gateway lumen further defining a gateway opening at an outer end of the gateway, said gateway outer end operably engageable with an input conduit for fluid communication with the gateway lumen.

11. The improved fluid circuit of claim 9 wherein the medial cylindrical inner surface within the medial portion is smaller than a diameter of a rearward inner cylindrical surface defined within the rearward portion, the member lumen further defining a forward frusto-conical transition surface located rearwardly of the forward cylindrical inner surface that transitions to the smaller diameter of the medial inner cylindrical surface of the member's medial portion, the member lumen further defining a rearward frusto-conical transition surface located forwardly of the rearward cylindrical inner surface that transitions to the smaller diameter of the medial inner cylindrical surface of the member's medial portion.

12. The improved fluid circuit of claim 11 wherein the at least one input conduit is in removable fluid communication with a fluid source via a removable connection located there-between, the fluid source selected from a group consisting of a syringe, an IV drip container, and an infusion pump.

13. The improved fluid circuit of claim 11 wherein an outward flow of fluid through the at least one input conduit is prevented by an occlusion device via a removable connection located there-between, the occlusion device selected from a group consisting of an occlusion cap and an inwardly-biased one-way valve.

14. The improved fluid circuit of claim 12 or 13 wherein the removable connection comprises a Luer lock.

15. The improved fluid circuit of claim 10 wherein a fluid flow through the member lumen is about equal to a fluid flow through the at least one pump.

16. The improved fluid circuit of claim 15 wherein the reduction in diameter of the medial cylindrical inner surface from the forward cylindrical inner surface defines a percentage reduction selected from a group consisting of about 25 percent and about 50 percent.

17. The improved fluid circuit of claim 11 further comprising at least one selectively occludable port defining a port lumen through the member and in fluid communication with the member lumen, and an inflatable tubular balloon defined within the port lumen, said port lumen further defining a port opening at an outer end of the port, and said tubular balloon selectively inflatable to selectively occlude the port lumen.

18. The improved fluid circuit of claim 17 wherein the at least one selectively occludable port is removably connected to the member.

19. A method of accessing a fluid circuit having at least one pump configured for fluid communication with a patient, and at least one oxygenator in fluid communication with the at least one pump and configured for fluid communication with the patient, the method comprising:
providing at least one access vessel located within the fluid circuit, said vessel comprising an axial member having forward, medial and rearward portions and defining a member lumen in fluid communication with the fluid circuit, said member lumen extending through the forward, medial and rearward portions of the member and defining a forward opening in the forward portion and a rearward opening in the rearward portion, the lumen further defining an inside diameter within the medial portion that is smaller than an inside diameter defined within both the forward and rearward portions of the member, said vessel further comprising at least one gateway defining a gateway lumen through the medial portion of the member and in fluid communication with the member lumen, said gateway lumen further defining a gateway opening at an outer end of the gateway, said gateway outer end operably engageable with an input conduit for fluid communication with the gateway lumen.

20. The method of claim 19 further comprising selectively occluding a port defining a port lumen through the member and in fluid communication with the member lumen.
